(19) United States
(12) Patent Application Publication
(10) Pub. No.: US 2023/0026427 A1
(43) Pub. Date: Jan. 26, 2023

(54) FETAL DECELLULARIZED NUCLEUS PULPOSUS MATERIAL AND METHODS FOR OBTAINING PHARMACEUTIC COMPOSITIONS TO BE USED IN THE TREATMENT OF INTERVERTEBRAL DISC DEGENERATION AND BACK PAIN

(71) Applicant: INEB - INSTITUTO DE ENGENHARIA BIOMEDICA, Porto (PT)

(72) Inventors: Mario Adolfo MONTEIRO DA ROCHA BARBOSA, Porto (PT); Joana CALDEIRA FERNANDES FREY RAMOS, Porto (PT); Raquel MADEIRA GONCALVES, Porto (PT); Morena FRANCESCA FIORDALISI, Porto (PT)

(73) Assignee: INEB - INSTITUTO DE ENGENHARIA BIOMEDICA, Porto (PT)

(21) Appl. No.: 17/783,875

(22) PCT Filed: Dec. 14, 2020
(86) PCT No.: PCT/PT2020/050049

§ 371 Date: Jun. 9, 2022

(30) Foreign Application Priority Data
Dec. 12, 2019 (PT) 116009
Dec. 10, 2020 (PT) 116929
Dec. 10, 2020 (PT) 116932

(51) Int. Cl. A61L 27/36; A61L 27/52; A61L 27/38

(52) U.S. Cl. CPC A61L 27/3658; A61L 27/3604; A61L 27/3687; A61L 27/52; A61L 27/3834; A61L 27/3856; A61L 2400/06; A61L 2430/38; A61L 2430/40

(57) ABSTRACT

A fetal-origin decellularized nucleus pulposus (NP) allogenic material to regenerate a host's Intervertebral Disc (IVD). The decellularized NP material, obtained from a vertebrate fetus and characterized by comprising high levels of collagen 12 and 14, is used in a pharmacological composition for the treatment of IVD degeneration. The advance is based on the increased ability of the fetal decellularized NP material to stimulate the host constituent cell's to increase the expression of collagen 2 and aggrecan, promoting intrinsic IVD regeneration. A related method includes preparing the pharmaceutical compositions of fetal decellularized material in the form of fragments/microparticles and hydrogel for an injectable mode of administration. The involved material, pharmaceutical compositions and methods may be advantageously used for the prevention and treatment of IVD degeneration and back pain in human and veterinary settings.

### TECHNICAL FIELD OF THE INVENTION

The present invention relates to the technical field of methods for preparing biomaterials; more specifically macromolecular materials or materials or having a macromolecular matrix.

### STATE OF THE ART

Intervertebral disc (IVD) degeneration occurs with age and is often the cause of low back pain, which affects 70-85% of the population.

Orthopedic surgical methods, such as spinal fusion, have been adopted to relieve mechanical back pain, but this is compromised by decreased spinal motion. In alternative, prosthetic/artificial disc implants may be used, with the inherent biocompatibility issues.

The IVD is composed of a layered annular fiber called annulus fibrosus (AF) and a jelly-like nucleus pulposus (NP) which contains constituent cells, such as chondrocytes, that produce collagen and proteoglycans.

The extracellular matrix (ECM) of the NP is mainly composed of aggrecan and type II collagen. The extracellular matrix of the AF is mainly composed of aggrecan and type I collagen.

It is known that the ECM of the IVD undergoes remodeling during normal ageing or in age-associated conditions that trigger the degenerative cascade and that this is accompanied by changes in the ECM protein composition (matrisome).

It has also been conventionally performed to directly inject collagen type II or collagen II-rich materials to replenish the damaged NP, as a means for treatment of IVD degeneration.

In this regard, Patent WO2019151444, for example, discloses therapeutic agent containing Low Adhesive Collagen obtained by enzymatically cutting a terminus of collagen, to promote the maintenance of intervertebral distance and the regeneration of NP cells and/or AF cells.

A number of other compositions are disclosed to harden the damaged NP and maintain disc height.

For example, Patent JP2019088900 discloses a composition for replenishing the NP, containing a monovalent metal salt of a low endotoxin alginic acid. The composition is applied to the NP site of a subject, hardens partially after application, and has fluidity when applied to the NP site.

Patent US2019117831 also discloses several polymer-based materials capable of forming a scaffold in situ at an IVD site. Patent US2018256784 discloses a decellularized adult tissue and biomaterials for use as grafts or in vitro cellular scaffolds, formed with the decellularized tissue, which may further comprise extrinsic cells to employ as a biomimetic of IVD tissue.

Overall, the therapies involving injection of matrix to the NP mostly employ compositions designed to provide structural support, or a scaffold graft to the IVD, or they are designed to replenish the ECM collagen, or cell components, using exogenous material or extrinsic cells.

However, the desired solution would be such that the employed materials would have the ability to promote the intrinsic regeneration of the IVD, through the stimulation of the host NP constituent cells to produce a renewed, regenerated ECM. Such solution and composition material with this property enhanced is still lacking in the prior art.

### SUMMARY OF THE INVENTION

The present invention refers to a biomaterial characterized by, comprising a fetal decellularized nucleus pulposus (NP) of the intervertebral disc (IVD) of a vertebrate animal, according to claim 1.

In another embodiment of the present invention, the said biomaterial is characterized by, comprising a quantity of collagen type XII (COL12A1) higher than 1.000.000 intensity-Based Absolute Quantification (iBAQ) units, defined by the sum of all peptide intensities divided by the number of theoretically observable tryptic peptides of a protein obtained by gel-free proteomics, most preferably a quantity higher than 10.000.000 COL12A1 iBAQ units and a ratio between Collagen type XII and total protein higher than 4 in comparison to young decellularized NP, according to claim 2.

In another embodiment, the said biomaterial is characterized by, comprising a quantity of collagen type XIV (COL14A1) higher than 1.400.000 iBAQ units, most preferably higher than 10.000.000 COL14A1 iBAQ units, and a ratio between Collagen type XIV and total protein higher than 10 in comparison to young decellularized NP, according to claim 3.

In another embodiment, the said fetus of a vertebrate animal comprises bovine fetus, porcine fetus, sheep fetus, horse fetus, donkey fetus, kangaroo fetus and other non-limiting examples of vertebrate fetus, according to claim 4.

Another embodiment of the present invention refers to a pharmaceutical composition for use in IVD regeneration characterized by, comprising the previously described biomaterial, according claim 5.

In another embodiment, the said pharmaceutical composition for use in IVD regeneration is characterized by, comprising the biomaterial in combination with other components, such as proteins, antibiotics, fungicides, preservation or culture medium, hydrogels, excipients, vehicle diluents, adjuvants, and combinations thereof, according to claim 6.

In another embodiment, the said a pharmaceutical composition for use in IVD regeneration is characterized by, comprising the said fetal decellularized biomaterial in the form of an implantable graft, for example an IVD graft, according to claim 7.

In another embodiment, the said pharmaceutical composition for use in IVD regeneration is characterized by, comprising the said fetal decellularized biomaterial in an injectable form, for example in the form of microfragments, according to claim 8.

In another embodiment, the said pharmaceutical composition for use in IVD regeneration is characterized by, comprising the said fetal decellularized biomaterial in an injectable form, for example in the form of a hydrogel, according to claim 9.

In another embodiment, the said pharmaceutical composition for use in IVD regeneration is characterized by, further comprising a other materials and a cell component, non-limiting examples include mesenchymal stem cells and exosomes, according to claim 10.

In another embodiment, the said pharmaceutical composition for use in IVD regeneration is characterized by, comprising COL12A1 and/or COL14A1 and combinations thereof, obtained from other natural or synthetic sources, according to claim 11.

The present invention also refers to a method to produce the biomaterial and the pharmaceutical composition in the form of injectable microparticles characterized by, comprising the steps of:
a) Obtaining a vertebrate fetus, most preferably a bovine fetus tail, most preferably male, most preferably 8 months of gestation.
b) Cleaning with ethanol 70%.
c) Removing excess fascia and muscle with a scalpel.
d) Cutting as close as possible to the vertebral body above and underneath to obtain the intervertebral disc.
e) Washing with phosphate buffered saline (PBS), most preferably supplemented with 10% Penicillin/Streptomycin and 1% Fungizone, for 15 minutes, under orbital agitation at 100 rpm.
f) Punching, most preferably using a 4 mm puncher, to obtain nucleus pulposus from the central zone of the disc.
g) Contacting the nucleus pulposus punches with a hypotonic buffer most preferably comprising 10 mM Tris-Base, 0.1% EDTA, 0.1% Gentamicin, 1% Penicillin/Streptomycin of and 0.5% of Fungizone at pH 7.8, most preferably for 18 h under orbital agitation at 165 rpm, at room temperature.
h) Removing hypotonic buffer and wash three times with PBS, most preferably for 1 hour under orbital agitation at 165 rpm, at room temperature.
i) Treating the punches most preferably for 1 hour with 0.1% SDS in 10 mM Tris-Base and 0.1% Gentamicin, 1% Penicillin/Streptomycin and 0.5% Fungizone at pH 7.8 under orbital agitation at 165 rpm, at room temperature.
j) Washing most preferably with 0.1% SDS in 10 mM Tris-Base and 0.1% Gentamicin, 1% Penicillin/Streptomycin and 0.5% Fungizone at pH 7.8, under orbital agitation at 165 rpm, at room temperature, for three times for 20 minutes each.
k) Performing a DNAse treatment, most preferably with a 20 mM Tris-Base, 2 mM MgCl₂, 0.1% Gentamicin, 1% Penicillin/Streptomycin and 0.5% Fungizone solution with, most preferably, 50 U/mL of DNAse, most preferably for 3 hours under orbital agitation at 165 rpm, at 37° C.
l) Washing with PBS 1×, most preferably 3 times, 20 minutes each, under orbital agitation (165 rpm), at room temperature.
m) Cutting decellularized IVD samples into pieces of 0.1-5 mm, most preferably 1 mm.
n) Submerging the pieces in an appropriate amount of an excipient or a vehicle solution, for example sterile saline.
o) Grinding the pieces with a tissue homogenizer, for example (Bertin Precellys 24, from Bertin Technologies) at 1000-10.000 rpms, preferably 6000 rpm for 5-60 seconds, preferably 30 seconds for 1-50 circles, preferably 20 circles at a temperature of 0-25° C., preferably 4° C.
p) Controlling the size of the decellularized fetal IVD-derived microparticles to be between 10-500 μm, most preferably 200 μm by filtering the suspension for example through an 80-mesh sample sieve (for 200 μm).
q) Adjusting the concentration of the microparticles suspension to 1-500 mg/ml, most preferably 50 mg/ml, with an appropriate amount of an excipient or a vehicle solution, for example sterile saline.
   according to claim 12.

The present invention also refers to a method to produce the biomaterial and the pharmaceutical composition in the form of a hydrogel characterized by, comprising the steps of:
a) Isolating and decellularizing fetal nucleus pulposus as described in claim 12 in steps a)-l).
b) Lyophilizing.
c) Cutting small pieces of 0.1-5 mm, most preferably 1 mm.
d) Solubilizing, most preferably to a concentration of 20 mg/mL, most preferably in 1 mg/mL pepsin in 3% acetic acid, most preferably at room temperature, most preferably for 72 hours.
e) Neutralizing to pH 7.4, most preferably using 0.1M sodium hydroxide.
f) Buffering with 10% of 10×PBS.
g) Maintaining the gels stable by submerging in 1×PBS.
   according to claim 13.

The present invention further refers to the use of the said a biomaterial and pharmaceutical composition as in vitro coating and scaffolds for repopulating, expanding and culturing cells, and extracellular matrix models, according to claim 14.

The present invention also refers to the use of the said biomaterial and pharmaceutical compositions for the prevention and treatment of degenerative disc disease and back pain in vertebrate animals including dogs and humans, according to claim 15.

The present invention also refers to the use of said biomaterial and pharmaceutical compositions for in the prevention and treatment of other degenerative conditions of cartilage tissues in animals, such as rheumatoid arthritis, osteoarthritis, cartilage rupture or detachment, achondroplasia, costochondritis, and polychondritis, according to claim 16.

### DETAILED DESCRIPTION OF THE INVENTION

The invention stems from the original and surprising discovery that fetal decellularized NP material from bovine intervertebral discs shows increased ability to stimulate the host constituent cell's to increase the expression of collagen 2 and aggrecan, both of which are key extracellular matrix components known to be lost during IVD degeneration in certain diseases with ageing. As such, the present invention refers to the use of a decellularized NP biomaterial from fetal origin for promoting intrinsic regeneration of IVDs.

In one embodiment of the present invention, the composition material is characterized by comprising a fetal biomaterial derived from the NP of a vertebrate fetus, a non-limiting example of which is a NP from IVDs of a mammalian fetus, for example a bovine fetus tail.

The said fetal NP biomaterial is further characterized by comprising a quantity of collagen type XII (COL12A1) higher than 1.000.000 intensity-Based Absolute Quantification (iBAQ) units, defined by the sum of all peptide intensities divided by the number of theoretically observable tryptic peptides of a protein obtained by gel-free proteomics, most preferably a quantity higher than 10.000.000 iBAQ units (FIG. 1A).

The said fetal NP material is further characterized by comprising a quantity of collagen type XIV (COL14A1) higher than 1.400.000 iBAQ units, most preferably higher than 10.000.000 iBAQ units (FIG. 1A).

Using other assessment methods, for example western-blot, the said fetal decellularized NP biomaterial can be further characterized by a ratio between Collagen type XII and total protein higher than 4 and the ratio between Collagen type XIV and total protein higher than 10, compared to decellularized young IVDs (FIG. 1B-C).

Another embodiment of the present invention refers to a pharmaceutical composition for IVD regeneration characterized by comprising the above mentioned fetal decellularized material in combination with other components, such as proteins, antibiotics, fungicides, preservation or culture medium, hydrogels, excipients, diluents, adjuvants, and combinations thereof.

In another embodiment of the present invention, the pharmaceutical composition for IVD regeneration is characterized by comprising the above mentioned fetal decellularized biomaterial in an injectable form, in microfragments or in a hydrogel.

In another embodiment of the present invention, the said composition of fetal decellularized material may further comprise a cell component such as mesenchymal stem cells, exosomes or other cells as an adjuvant for cell therapy of IVD degeneration.

In another embodiment of the present invention, the composition for IVD regeneration is characterized by comprising COL12A1 and/or COL14A1, and combinations thereof, obtained from other natural or synthetic sources.

The optimal conditions for decellularization of fetal IVDs to achieve lowest levels of DNA and highest level of glycosaminoglycans were assessed (FIG. 2). As such, in another embodiment of the present invention a method to produce the said bovine NP decellularized biomaterial can be developed, the said method comprising the steps of:
1. Obtaining fetus (most preferably male; most preferably 8 months of gestation) bovine tails and transport on ice to the lab.
2. Cleaning the tails with ethanol 70%.
3. Removing excess fascia and muscle from the tails with a scalpel.
4. By using a sterile scalpel cutting through the intervertebral disc (IVD) as close as possible to the vertebral body above and underneath the disc to obtain the disc as complete as possible (without endplate).
5. Freezing the isolated discs in liquid nitrogen and 2-methylbutane.
6. Storing at −80° C. until further use.
7. Inside the flow chamber, unfreezing fetal intervertebral disc at room temperature and wash with PBS 1×, supplemented with 10% Pen/Strep and 1% Fungizone for 15 minutes, under orbital agitation (100 rpm).
8. After wash, by the help of a 4 mm punch, obtaining the nucleus pulposus from the central zone of the intervertebral disc.
9. Cutting in half the nucleus pulposus in order to obtain pieces with similar height.
10. Transferring it into a 24-well plate with 1 mL of Hypotonic buffer A for 18 h under orbital agitation (165 rpm) at room temperature.
11. After 18 h, removing Hypotonic Buffer A and wash three times with 1 mL of PBS. Each wash is for 1 hour under orbital agitation (165 rpm) at room temperature.
12. Preparing SDS 0.1% solution in Hypotonic Buffer B (1 mL/sample) and treating for 1 hour, under orbital agitation (165 rpm) at room temperature.
13. Washing three times with 1 mL of Hypotonic Buffer B. Each wash is for 20 minutes under orbital agitation (165 rpm) at room temperature.
14. Preparing DNAse treatment solution, by adding DNAse I to the solution (50 U/mL). Adding 1 mL of this solution to each well and start the treatment for 3 hours under orbital agitation (165 rpm) at 37° C.
15. Washing three times with 1 mL of PBS 1×. Each wash is for 20 minutes, under orbital agitation (165 rpm), at room temperature.

The said solutions in the above-mentioned method are characterized by comprising the compositions described in the following table:

TABLE 1
Solutions used in NP decellularization

| Solution | Composition | pH |
|---|---|---|
| PBS 1X | Phosphate | 7.4 |
| | Buffered saline | |
| Hypotonic | 10 mM Tris-Base | 7.8 |
| Buffer A | 0.1% EDTA | |
| Hypotonic | 10 mM Tris-Base | 7.8 |
| Buffer B | | |
| DNAse | 20 mM Tris-Base | 7.8 |
| Treatment | 2 mM MgCl₂ | |

Furthermore, at the moment of use, all the solutions are supplemented with 0.1% of Gentamicin, 1% of Penicillin/Streptomycin and 0.5% of Fungizone, to avoid contaminations.

In another embodiment, after decellularization, the decellularized NP is equilibrated overnight in IVD-medium in a hypoxia incubator (37° C., 6% O₂ and 8.5% CO₂) to be used in vitro as scaffold for repopulating and culturing cells.

The said IVD media comprises the following components:
DMEM low glucose
NaHCO₃
Penicillin/Streptomycin
Fungizone
NaCl/KCl solution
Fetal Bovine Serum
Distilled water

For preparing an injectable form of the above mentioned decellularized biomaterial, the following additional steps are comprised:
1. Cut decellularized IVD samples into pieces of 0.1-5 mm, most preferably 1 mm.
2. Submerge the pieces in an appropriate amount of an excipient or a vehicle solution, for example sterile saline.
3. Grind the pieces with a tissue homogenizer, for example (Bertin Precellys 24, from Bertin Technologies) at 1000-10.000 rpms, preferably 6000 rpm for 5-60 seconds, preferably 30 seconds for 1-50 circles, preferably 20 circles at a temperature of 0-25° C., preferably 4° C.
4. Control the size of the decellularized fetal IVD-derived microparticles to be between 10-500 μm, most preferably 200 μm by running the suspension for example through an 80-mesh sample sieve (for 200 μm).
5. Adjust the concentration of the decellularized fetal IVD-derived microparticles suspension to 1-500 mg/ml, most preferably 50 mg/ml.

When the vertebrate fetal NP material obtained by the IVD decellularization method described above is put into contact with adult NP cells, it surprisingly demonstrates the increased ability to stimulate the expression of collagen 2 and aggrecan by these cells, with an observed significant increase in collagen 2 and aggrecan mRNA levels and protein immunostaining (FIG. 3A-E).

Furthermore, rheological analysis has shown that fetal decellularized NPs have distinct structural and biochemical properties, being less stiff, as demonstrated by lower complex shear modulus (G*) values (at 5% of strain) than young-derived scaffolds, retrieved from the linear viscoelastic region (0.04-1 Hz) of the frequency sweep (FIG. 4). Also, collagen organization and architecture in general is distinct, as assessed by Picrosirius red staining followed by polarized light microscopy (FIG. 5A-B). Results show increased red to green ratio fibers which indicate more mature and thicker fibers, mainly composed of collagen type I.

In another embodiment of the present invention, the fetal IVDs material may be injectable through the form of a hydrogel. The parameters of a method to produce a Fetal IVD-derived hydrogel were addressed. Decellularized NPs (dNPs) were lyophilized for hours. After lyophilization, dNPs pooled for digestion. Samples (with or without chopping) were suspended at 20 mg/mL in mg/mL pepsin in 3% acetic acid, 0.1 M or 0.01 M of hydrochloric. dNPs were then placed on a stir plate at 37° C. or at room temperature from 24 to 72 hours to facilitate digestion. After this time, pre-gel solutions were neutralized (to pH 7.4) using 0.1M sodium hydroxide and buffered with 10% of 10× sterile PBS. Solubilized dNPs were stored at 4° C. for up to 1 month until use. All the conditions tested have been summarized in Table 2.

TABLE 2
Table specifying all the parameters tested for the hydrogel formation

| | | | | Acidic | | Digestion |
|---|---|---|---|---|---|---|
| Age | Cuting | \|Tissue\| | \|Protease\| | solution | Temperature | time |
| Fetus | Yes | 20 mg/ml | 1 mg/ml | O.1M HCL | RT | 64 h* |
| Young | No | | | 3% AA | 37 °C. | 72 h* |
| | | | | 0.01M HCL | | |

Legend: Y—Young; F—Fetus; |Tissue|—Tissue concentration; |Protease|—Protease concentration; Temp—temperature. 64 h* 72 h* 64 hours and 72 hours were the time points used for further hydrogel characterization. However, the procedure included other time points: 24 hours, 32 hours and 48 hours.

a) Water Retention

All samples were lyophilized prior to solubilization. Both their wet (prior to decellularization) and dry weights were registered for further reference. There are significant statistical differences in water percentage amount between the two types of NPs. Water content was determined gravimetrically by measuring a sample's wet weight and then their corresponding dry weight following lyophilization. Water percentage was calculated by dividing this difference by the wet weight. Interestingly, fetal NPs seem to retain more water than young ones. (FIG. 6).

b) Gelification

Collagen thermal gelation occurs through monomer aggregation and self-assembly into thin filaments that crosslink into collagen fibers contributing to hydrogel formation. Concomitantly, the absorbance at 405 nm increases. As such, the turbidimetric gelation kinetics of the pre-gel solutions that were effectively solubilized and presented a hydrogel-like behavior was further characterized spectrophotometrically (FIG. 7). Stabilization of absorbance of the pre-gels occurred after around 20 minutes, indicating near complete gelation, but only for hydrogels at 20 mg/mL concentration solubilized using pepsin (1 mg/ml concentration) in acetic acid 3% solutions at 37° C. for 72 h. All the other conditions, including negative controls (acetic acid and HCl solution) and water, did not seem to gelate. Comparing fetal-based and young-based hydrogels that achieved complete gelation, it was observed that fetal-based formulations presented higher absorbance values (fetus 1.025 nm value compared to 0.614 in young formulations).

c) Viscoelastic Properties (Stiffness)

By comparing the values of the complex shear modulus (G*) obtained from the LVR, we surprisingly observed that fetus hydrogels are stiffer than young (G*˜184 Pa vs. G*˜130.5 Pa, respectively, FIG. 8).

In conclusion, the optimal parameters for the creation of a dNP-based hydrogel comprise the steps of:
Lyophilization.
Cutting to small pieces of 0.1-5 mm, most preferably 1 mm.
Solubilizing fetal dNPs at a concentration of 20 mg/mL in 1 mg/mL pepsin in 3% acetic acid at room temperature for 72 hours.
Neutralization to pH 7.4 using 0.1M sodium hydroxide.
Buffering with 10% of 10×PBS.
Maintained the gels submerged in 1×PBS.

We ascertained that hydrogels are stable in PBS for at least 7 days.

The present invention also refers to the use of the said material and hydrogel in a treatment to slow, halt or reverse IVD degeneration and back pain, including neck, cervical and back pain.

In another embodiment of the present invention the said collagen type 12 and type 14-rich material and hydrogel may be obtained through a mixture of synthetic collagen type 12 and collagen type 14, and combinations thereof.

In summary, through the constituents and properties, specifically related to the fetal origin, the biomaterial, pharmaceutical compositions and methods to produce injectable microparticle and hydrogel forms of the present invention may be advantageously used for preventing and treating IVD degeneration and back pain (including neck, cervical and back pain) in vertebrate animals, including dogs and humans, and for preventing and treating degenerative conditions of cartilage tissues other than the intervertabral disc, such as rheumatoid arthritis, osteoarthritis, cartilage rupture or detachment, achondroplasia, costochondritis, and polychondritis.

### BRIEF DESCRIPTION OF THE FIGURES

FIG. 1: Collagen 12 and 14 composition of fetal and young age decellularized nucleus pulposus material.

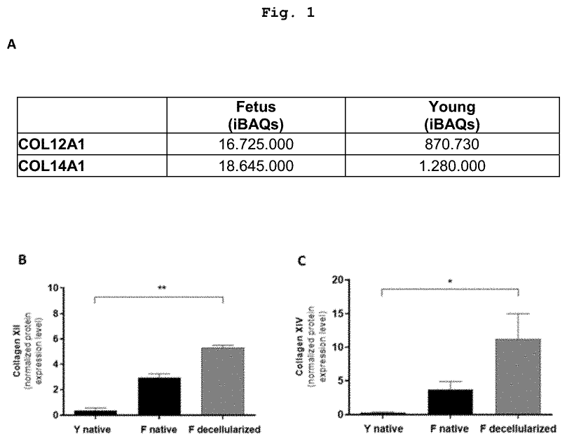

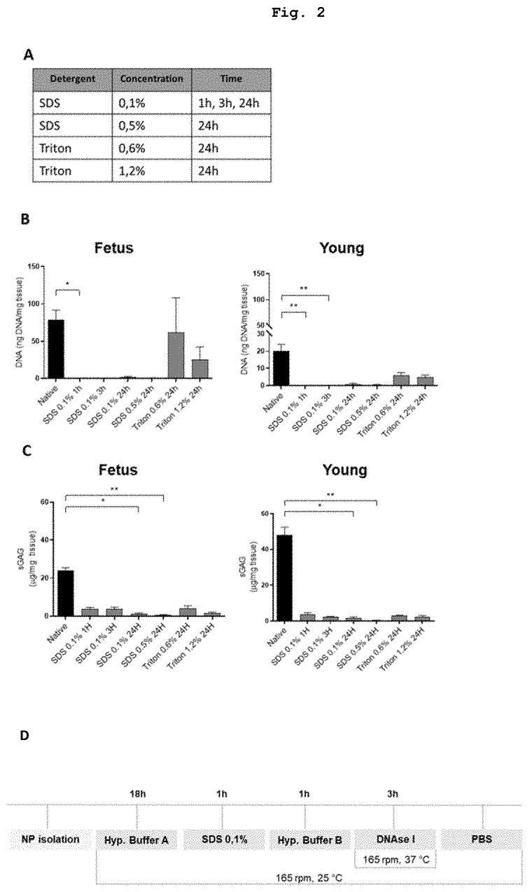

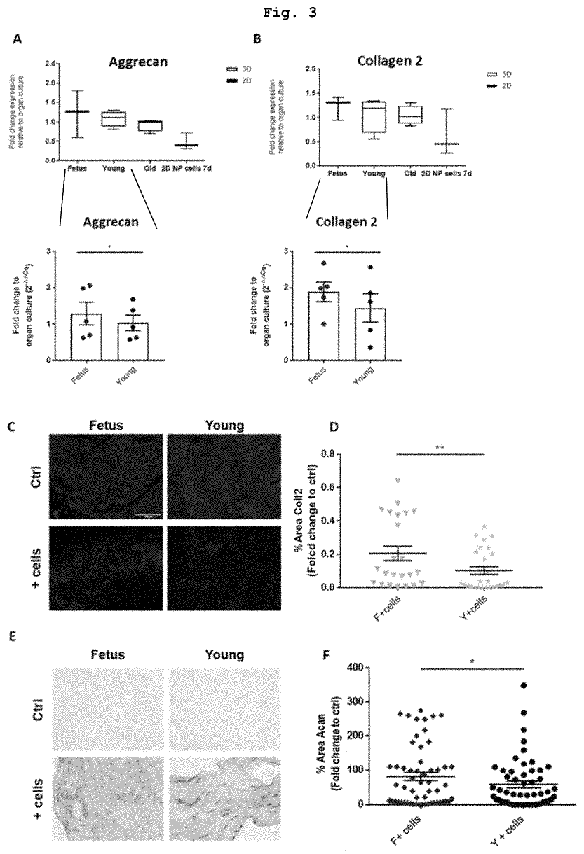

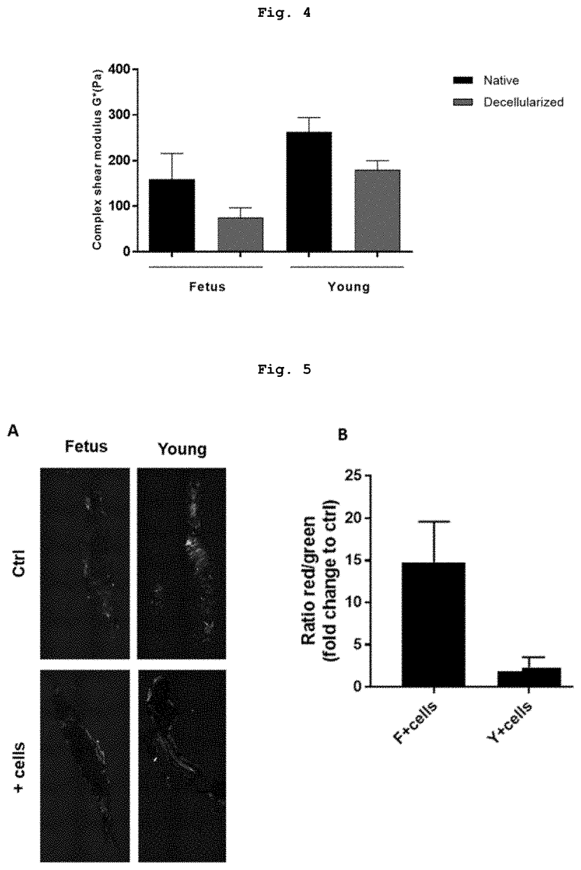

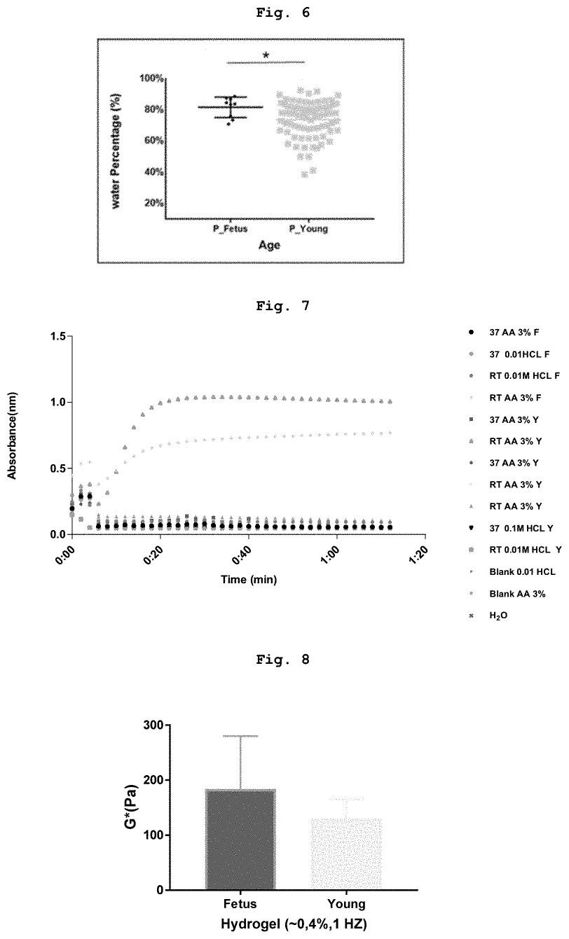

(A) Intensity-Based Absolute Quantification (iBAQ) units are defined by the sum of all peptide intensities divided by the number of theoretically observable tryptic peptides of a protein obtained by intensity-Based Absolute Quantification (iBAQ) units, defined by the sum of all peptide intensities divided by the number of theoretically observable tryptic peptides of a protein obtained by proteomic gel-free proteomics. The absolute quantity of collagen 12 and 14 in decellularized bovine IVD scaffolds is 19 and 15-fold higher, respectively, in fetal NP relative to young-derived NP. Western blotting for Collagen type XII (B) and Collagen type XIV (C) of fetus (F) native and decellularized NPs and compared to the young (Y) native NPs (negative control). Graphs represents the average of three to four independent experiments obtained by band quantification. Protein expression levels were normalized by the total protein loading. Data are expressed as mean±SEM. Kruskal Wallis test followed by Dunn's multiple comparison test. As observed from the graphs, ratio between Collagen type XII and total protein in fetal decellularized is higher than 4 and the ratio between Collagen type XIV and total protein is higher than 10, compared to young.

FIG. 2. Decellularization efficiency of several experimental methods using bovine nucleus pulposus from young and fetal donors. Chemical detergents investigated for bovine nucleus pulposus decellularization. Sodium dodecyl sulfate (SDS) and Triton X-100 (Triton) treatments were explored at different concentration and time point (A). PicoGreen DNA quantification (B) and Blyscan sulfate GAGs quantification (C) of native and decellularized fetal and young NPs from two to three independent experiments. Data were normalized by wet weight (ng/mg for DNA and μg/mg for GAGs). Data are expressed as mean±SEM. Kruskal Wallis test followed by Dunn's multiple comparison test. Data from each decellularization treatment and age group were compared to the correspondent control (native). *p<0.05; **p<0.01. D—Schematic representation of optimal chemical, mechanical and enzymatic decellularization treatments.

FIG. 3: Molecular evaluation of bovine nucleus pulposus from different ages after repopulation. Constituent cells from young adult IVDs were isolated through a method comprising the following steps:
1. Obtain young adult bovine tails (male ˜12 months old) within 2-3 h after animal sacrifice, from the local abattoir and dissect aseptically. Wash the tails with EtOH 70% before put inside the flow chamber.
2. Remove excess fascia and muscle with a scalpel.
3. By using a sterile scalpel cut through the intervertebral disc (IVD) as close as possible to the vertebral body above and underneath the disc to obtain the disc as complete as possible.
4. By using a scalpel blade, separate the nucleus pulposus from the annulus fibrosus.
5. Weight the isolated nucleus pulposus. While dissecting keep the discs hydrated with isolation media on a sterile Petri dish.
6. Cut the isolated nucleus pulposus into approximately 2×2 mm segments with blade.
7. Use 10% of the nucleus pulposus wet weight as a volume of digestion media.
8. Weight collagenase I (0.5 mg/mL) and add the corresponding volume of digestion media. Add also DNAse I and filter the digestion media.
9. Transfer the tissue in the falcon with the digestion media and a sterile magnet.
10. Incubate the tissue overnight at 37° C. on a magnetic agitator (gentle agitation) in hypoxia incubator.
11. After digestion, filter through a 40/70 μm cell strainer to remove undigested ECM and produce a single cell suspension.
12. Centrifuge the filtrate at 400 g for 15 minutes in a 15 mL falcon to get a cell pellet and clear suspension.
13. Remove the supernatant and re-suspend the cells in 5 mL of IVD-medium.
14. Remove 10 μL of the suspension (homogenize well) for cell counting and mix with 10 μL of trypan blue. Count total cells using a microscope and haemocytometer to give an estimate of total cell number in 5 mL.
15. Freeze some aliquot of fresh cells in Trizol to use for RNA extraction (eventually as a control for gene expression).
16. Maintain in 2D culture other fresh cells in hypoxia incubator with IVD media to use for RNA extraction (as a control for gene expression).

Afterwards, the isolated cells were used for repopulation of decellularized nucleus pulposus material from different ages, including fetal and young, employing the following steps:
1. After equilibration of decellularized nucleus pulposus and isolation of adult bovine nucleus pulposus cells, start with cell seeding by dropping and scaffold turnover.
2. Resuspend 1×10⁵ cells in 10 ul of IVD media (5 ul each side).
3. Drop 5 ul of cell suspension in one side of the decellularized matrix and incubate for 2 hours in hypoxia atmosphere without IVD media.
4. After 2 hours, turn the nucleus pulposus and drop the other 5 ul of cell suspension. Incubate in hypoxia atmosphere for 2 hours without IVD media.
5. After these 4 hours, add IVD media and maintain the scaffolds in ex vivo culture for 7 days in hypoxia atmosphere.
6. Use as a control decellularized nucleus pulposus from different ages without cell seeding, cultured for 7 days as the repopulated matrices.
7. Collect and store conditioned media every 2 days.
8. Measure metabolic activity by Resazurin assay 24 h, 3 days, 7 days after cell seeding.

After 7 days of culture samples were processed for histology and for gene expression, according to the following procedures:
1. Histology: fix the scaffolds in formalin overnight at 4 degree. The day after mount the cassettes with the sample and leave in PBS until the use for Tissue Processor and Embedding.
2. Gene expression: cut the repopulated nucleus pulposus by the help of blades and digest with Pronase at 37 degree under magnetic agitation for 1 hour. Neutralize the enzyme activity with FBS and wash tissue with cold PBS. Freeze the tissue with liquid nitrogen and store at −80 degree until further use. Afterwards proceed with RNA extraction and collagen 2 mRNA quantification by real-time PCR.

mRNA expression level of aggrecan (A) and collagen type II (B) by quantitative real-time PCR, of fetus, young and old repopulated NPs, compared to 2D bovine NP cells, after 7 days of ex vivo culture. mRNA values were interpolated in a calibration curve (mRNA level of 2D bovine NP cells at different concentrations) and normalized by GAPDH, an internal control (mRNA level of 2D bovine NP cells) and native bovine NP (mRNA level of organ culture: 8 mm punched NP cultured ex vivo for 7 days). Data are represented as box and whiskers plots. Error bars on box-and-whiskers plots indicate the minimum and maximum values. Kruskal-Wallis Test followed by Dunn's multiple comparison test.

Collagent type II composition of bovine nucleus pulposus from different ages after repopulation: Expression of collagen by immunofluorescence (C) in fetus (F+cells) and young (Y+cells) repopulated NPs, compared to the correspondent controls (decellularized matrices; ctrl). Representative images of four to six independent experiments. Collagen type II: magnification 20× and scale bar 100 μm. Collagen type II quantification by IntensityStatisticsMask Software (D). Data are represented as dot plots. Error bars plots indicate the minimum and maximum values. Graphs corresponds to the mean with SEM of the technical replicates. Wilcoxon test was used in comparisons.

Aggrecan content of bovine nucleus pulposus from different ages after repopulation: Expression of aggrecan by immunoistochemistry (E) in fetus (F+cells) and young (Y+cells) repopulated NPs, compared to the correspondent controls (decellularized matrices; ctrl). Representative images of four to six independent experiments. Aggrecan: magnification 20×. Quantification by ImageJ Software (F). Data are represented as dot plots. Error bars plots indicate the minimum and maximum values. Graphs corresponds to the mean with SEM of the technical replicates. Wilcoxon test was used in comparisons.

FIG. 4. Structural and biochemical composition of bovine nucleus pulposus from fetal and young decellularized NPs with the optimal procedure.

A. Biomechanical characterization of bovine nucleus pulposus from different ages decellularized with the optimal procedure (SDS 0.1% 1h). Complex shear modulus (G*) values (at 5% of strain), retrieved from the linear viscoelastic region (0.04-1 Hz) of the frequency sweep, performed by rheology, of fetus and young native and decellularized NPs. Graphs represents the average of three independent experiments (three to four NPs tissue from the same animal donor for each native and decellularized condition). Data are expressed as mean±SEM. Kruskal-Wallis test followed by Dunn's multiple comparison test.

FIG. 5. Picrosirius red staining followed by polarized light microscopy to evaluate collagen organization (A). Graph (B) represents the average ratio of green to red fibers of four to eight NPs for each native and decellularized condition. Data are expressed as mean±SEM. Mann-Whitney test was used in comparisons.

FIG. 6—The graph shows the percentage of water lost per sample following freeze drying.

FIG. 7—Turbidimetric gelation kinetics. Representative curves of the different NP-derived hydrogel compositions tested, as well as of the controls. Neutralized and buffered pre-gel solutions were added to 96-well plates at 37° C. to induce gelation. The absorbance was measured every 2 minutes at 405 nm.

FIG. 8—The graph presents the values of the mean G* for each age expressed as mean±SEM.

### OTHER EXAMPLES

Examples of other forms of the present invention comprise the use of nucleus pulposus material derived from the fetus of other vertebrates, including, but not limited to, decellularized nucleus pulposus material from porcine fetus, decellularized nucleus pulposus material from sheep fetus, decellularized nucleus pulposus material from horse fetus, decellularized nucleus pulposus material from donkey fetus, decellularized nucleus pulposus material from kangaroo fetus etc.
Lisbon, 14 Dec. 2020

1. A biomaterial characterized by, comprising a fetal decellularized nucleus pulposus (NP) of the intervertebral disc (IVD) of the fetus of a vertebrate animal.

2. The biomaterial according to claim 1 characterized by, comprising a quantity of collagen type XII (COL12A1) higher than 1.000.000 intensity-Based Absolute Quantification (iBAQ) units, defined by the sum of all peptide intensities divided by the number of theoretically observable tryptic peptides of a protein obtained by gel-free proteomics, most preferably a quantity higher than 10.000.000 COL12A1 iBAQ units and a ratio between Collagen type XII and total protein higher than 4 in comparison to young decellularized NP.

3. The biomaterial according to claim 1 characterized by, comprising a quantity of collagen type XIV (COL14A1) higher than 1.400.000 iBAQ units, most preferably higher than 10.000.000 COL14A1 iBAQ units and a ratio between Collagen type XIV and total protein higher than 10 in comparison to young decellularized NP.

4. The biomaterial according to claim 1 in which the said fetus of a vertebrate animal comprises bovine fetus, porcine fetus, sheep fetus, horse fetus, donkey fetus, kangaroo fetus and other non-limiting examples of vertebrate fetus.

5. A pharmaceutical composition for use in IVD regeneration characterized by, comprising the said biomaterial, as described in claim 1.

6. A pharmaceutical composition for use in IVD regeneration according to claim 5 characterized by, comprising the biomaterial in combination with other components, such as proteins, antibiotics, fungicides, preservation or culture medium, hydrogels, excipients, vehicle diluents, adjuvants, and combinations thereof.

7. A pharmaceutical composition for use in IVD regeneration according to claim 5 characterized by, comprising the said fetal decellularized biomaterial in the form of an implantable graft, for example an IVD graft.

8. A pharmaceutical composition for use in IVD regeneration according to claim 5 characterized by, comprising the said fetal decellularized biomaterial in an injectable form, for example in the form of microparticles.

9. A pharmaceutical composition for use in IVD regeneration according to claim 5 characterized by, comprising the said fetal decellularized biomaterial in an injectable form, for example in the form of a hydrogel.

10. A pharmaceutical composition for use in IVD regeneration according to claim 5 characterized by, further comprising other materials and cell component, non-limiting examples include cells, mesenchymal stem cells and exosomes.

11. A pharmaceutical composition for use in IVD regeneration characterized by, comprising COL12A1 and/or COL14A1 and combinations thereof, obtained from other natural or synthetic sources.

12. A method to produce the biomaterial and the pharmaceutical composition in the form of injectable microparticles according to claim 7 characterized by, comprising the steps of:
a) Obtaining a vertebrate fetus, most preferably a bovine fetus tail, most preferably male, most preferably 8 months of gestation.
b) Cleaning with ethanol 70%.
c) Removing excess fascia and muscle with a scalpel.
d) Cutting as close as possible to the vertebral body above and underneath to obtain the intervertebral disc.
e) Washing with phosphate buffered saline (PBS), most preferably supplemented with 10% Penicillin/Streptomycin and 1% Fungizone, for 15 minutes, under orbital agitation at 100 rpm.
f) Punching, most preferably using a 4 mm puncher, to obtain nucleus pulposus from the central zone of the disc.
g) Contacting the nucleus pulposus punches with a hypotonic buffer most preferably comprising 10 mM Tris-Base, 0.1% EDTA, 0.1% Gentamicin, 1% Penicillin/Streptomycin of and 0.5% of Fungizone at pH 7.8, most preferably for 18 h under orbital agitation at 165 rpm, at room temperature.
h) Removing hypotonic buffer and wash three times with PBS, most preferably for 1 hour under orbital agitation at 165 rpm, at room temperature.
i) Treating the punches most preferably for 1 hour with 0.1% SDS in 10 mM Tris-Base and 0.1% Gentamicin, 1% Penicillin/Streptomycin and 0.5% Fungizone at pH 7.8 under orbital agitation at 165 rpm, at room temperature.
j) Washing most preferably with 0.1% SDS in 10 mM Tris-Base and 0.1% Gentamicin, 1% Penicillin/Streptomycin and 0.5% Fungizone at pH 7.8, under orbital agitation at 165 rpm, at room temperature, for three times for 20 minutes each.
k) Performing a DNAse treatment, most preferably with a 20 mM Tris-Base, 2 mM MgCl₂, 0.1% Gentamicin, 1% Penicillin/Streptomycin and 0.5% Fungizone solution with, most preferably, 50 U/mL of DNAse, most preferably for 3 hours under orbital agitation at 165 rpm, at 37° C.
l) Washing with PBS 1×, most preferably 3 times, 20 minutes each, under orbital agitation (165 rpm), at room temperature.
m) Cutting decellularized IVD samples into pieces of 0.1-5 mm, most preferably 1 mm.
n) Submerging the pieces in an appropriate amount of an excipient or a vehicle solution, for example sterile saline.
o) Grinding the pieces with a tissue homogenizer, for example (Bertin Precellys 24, from Bertin Technologies) at 1000-10.000 rpms, preferably 6000 rpm for 5-60 seconds, preferably 30 seconds for 1-50 circles, preferably 20 circles at a temperature of 0-25° C., preferably 4° C.
p) Controlling the size of the decellularized fetal IVD-derived microparticles to be between 10-500 μm, most preferably 200 μm by filtering the suspension for example through an 80-mesh sample sieve (for 200 μm).
q) Adjusting the concentration of the microparticles suspension to 1-500 mg/ml, most preferably 50 mg/ml, with an appropriate amount of an excipient or a vehicle solution, for example sterile saline.

13. A method to produce the biomaterial and the pharmaceutical composition in the form of a hydrogel according to claim 8 characterized by, comprising the steps of:
a) Isolating and decellularizing fetal nucleus pulposus as described previously in steps a)-l).
b) Lyophilizing.
c) Cutting small pieces of 0.1-5 mm, most preferably 1 mm.
d) Solubilizing, most preferably to a concentration of 20 mg/mL, most preferably in 1 mg/mL pepsin in 3% acetic acid, most preferably at room temperature, most preferably for 72 hours.
e) Neutralizing to pH 7.4, most preferably using 0.1M sodium hydroxide.
f) Buffering with 10% of 10×PBS.
g) Maintaining the gels stable by submerging in 1×PBS.

14. A biomaterial and pharmaceutical composition as described in claim 1 for use as in vitro coating and scaffolds for repopulating, expanding and culturing cells, and extracellular matrix models.

15. A biomaterial and pharmaceutical composition as described in claim 1 for use in the prevention and treatment of pathologic and age-related degenerative disc disease and back pain, including neck, cervical and back pain, in vertebrate animals including dogs and humans.

16. A biomaterial and pharmaceutical composition as described in claim 1 for use in the prevention and treatment of other degenerative conditions of cartilage tissues in animals, such as rheumatoid arthritis, osteoarthritis, cartilage rupture or detachment, achondroplasia, costochondritis, and polychondritis.
